(19) United States
(12) Patent Application Publication
(10) Pub. No.: US 2025/0144011 A1
(43) Pub. Date: May 8, 2025

(54) MICRONEEDLE FORMULATION, MICRONEEDLE PATCH, AND PREPARATION METHODS THEREOF

(71) Applicants: YOUWE (ZHUHAI) BIOTECHNOLOGY CO., LTD, ZHUHAI (CN); CREWAY (ZHUHAI) PHARMACEUTICAL TECHNOLOGY CO., LTD, ZHUHAI (CN)

(72) Inventors: Yonghao MA, ZHUHAI (CN); Jiacheng LUO, ZHUHAI (CN); Chengguo LI, ZHUHAI (CN); Gang LENG, ZHUHAI (CN)

(21) Appl. No.: 18/832,426

(22) PCT Filed: Nov. 23, 2022
(86) PCT No.: PCT/CN2022/133730

§ 371 Date: Jul. 23, 2024

(30) Foreign Application Priority Data
Jun. 2, 2022 (CN) 202210625725.4

(51) Int. Cl. A61K 9/00; A61K 8/02; A61K 8/34; A61K 8/49; A61K 8/67; A61K 31/445; A61K 31/573; A61K 31/704; A61K 38/26; A61K 38/27; A61K 38/29; A61K 39/00; A61K 39/145; A61K 39/215; A61K 39/29; A61Q 19/08

(52) U.S. Cl. CPC A61K 9/0021; A61K 8/0208; A61K 8/347; A61K 8/4953; A61K 8/498; A61K 8/671; A61K 8/675; A61K 8/678; A61K 31/445; A61K 31/573; A61K 31/704; A61K 38/26; A61K 38/27; A61K 38/29; A61K 39/145; A61K 39/215; A61K 39/292; A61Q 19/08; A61K 2039/53; A61K 2800/91

(57) ABSTRACT

The present disclosure discloses a microneedle formulation, a microneedle patch, and preparation methods thereof. The microneedle formulation includes the following components in mass percentage: 15-55% of microneedle scaffold material, 0.05-5% surfactant with cyclic structure, 0-15% of active components, and the remaining being deionized water. The microneedle patch is prepared from the aforementioned microneedle formulation.

### FIELD OF THE DISCLOSURE

The present disclosure generally relates to the field of microneedle technology and, more particularly, relates to microneedle formulation, microneedle patch, and preparation method thereof.

### BACKGROUND

Microneedle is a novel transdermal drug delivery method that significantly enhances the efficiency of transdermal drug delivery. Theoretically, it can achieve a transdermal efficiency of up to 100%, offering the advantage of high efficiency in small dosages. As a new type of transdermal drug delivery product, its production process is entirely new and needs continuous improvement. Process optimization may help increase product yield, improve factory production efficiency, and reduce material costs, labor costs, and time costs.

Mold leveling for microneedle production is a commonly used manufacturing process due to its high efficiency and product stability. However, during the production process of this method, air bubbles inside the material of the mold are easy to aggregate and rupture, causing the solution to aggregate into droplets and thus form voids. The voids may expand to the edge of the mold, due to the aggregation and contraction characteristics of the liquid itself, eventually leading to the production of defective products.

In general manufacturing processes, centrifugation and vacuuming methods are used to eliminate small bubbles in soluble microneedle formulations. However, during the material filling and static drying processes (due to the presence of bubbles in the small holes of the silicone mold), bubbles may also be introduced, resulting in a high defect rate for certain formulations. At the same time, for soluble microneedle formulations that require the addition of poorly soluble or insoluble active components, the pressure for increased defect rates faced by microneedle products is even greater.

Optimizing the formulation of microneedle preparations and improving the stability and yield rate of the production process of microneedle products are of crucial significance to the development of enterprises.

### SUMMARY OF THE DISCLOSURE

To address the aforementioned technical issues, the present disclosure provides a microneedle formulation, microneedle patch, and their preparation methods. The present disclosure utilizes a combination of surfactant with cyclic structure with microneedle scaffold material to enhance the stability of bubbles in microneedle formulations, increase the solubility of active components in microneedle, thereby improving the stability of the production process and product yield rate.

To achieve the above objectives, the present disclosure employs the following technical solutions:

A microneedle formulation including the following components in mass percentages:
15-55% of microneedle scaffold material;
0.05-5% of surfactant with cyclic structure; and
the remaining being deionized water.

Preferably, the microneedle formulation includes the following components in mass percentages:
15-50% of microneedle scaffold material;
1-5% of surfactant with cyclic structure; and
the remaining being deionized water.

Furthermore preferably, the microneedle formulation includes the following components in mass percentages:
20-27% of microneedle scaffold material;
1.5-4% of surfactant with cyclic structure; and
the remaining being deionized water.

Preferably, the surfactant with cyclic structure is at least one of a pyridinium salt, a morpholinium salt, an imidazolinium salt, a piperazinium salt, and a saponin.

Specifically, the pyridinium salt is at least one of a pyridone ethanolamine salt, pyridine hydrochloride, and 2,6-diaminopyridine sulfate; the morpholinium salt is soybean oil ethylmorpholinium ethyl sulfate and/or isostearamidopropylmorpholine lactate salt; the piperazinium salt is hydroxyethyl piperazine ethane sulfonate and/or p-chlorophenylpiperazine hydrochloride; the imidazolinium salt is isostearylethyl imidazolinium ethyl sulfate; and the saponin is at least one of soapberry saponin, ginsenoside, astragalus saponin, aesculus saponin, diosgenin, and ruscogenin;

Where, the astragalus saponin includes but is not limited to astragalus saponin I, II, III, IV, V, VI and VII.

Preferably, the microneedle scaffold material is at least one of sodium hyaluronate, polyvinylpyrrolidone, chondroitin sulfate, sodium hydroxyethyl cellulose, collagen, sucrose, trehalose, maltose, chitosan, and dextran; further preferably, sodium hyaluronate.

Further preferably, the microneedle scaffold material contains a large molecule microneedle scaffold material and a small molecule microneedle scaffold material in a mass ratio of 1:1-3:1, or in a mass ratio of 0:1-0.5:1 for the large molecule microneedle scaffold material and the small molecule microneedle scaffold material.

Further preferably, the molecular weight of the large molecule microneedle scaffold material is ≥50,000 Da; and the molecular weight of the small molecule microneedle scaffold material is <50,000 Da.

The present disclosure also provides a preparation method for a microneedle formulation, including the following steps: dissolving the aforementioned formulation amount of microneedle scaffold material and surfactant with cyclic structure in deionized water, stirring uniformly, and degassing, to obtain the microneedle formulation.

Preferably, the degassing process includes, but is not limited to, vacuum and/or centrifugation.

In other preferred embodiments of the present disclosure, the microneedle formulation may also contain an active component with a mass percentage not exceeding 15%.

In other preferred embodiments of the present disclosure, the microneedle formulation contains a skincare component; the skincare component includes at least one of the following: a whitening component, a moisturizing component, an antioxidant component, an eye bag removing component, an anti-aging component, and a vascular color reducing component.

Preferably, the whitening component includes at least one of glabridin, niacinamide, phloretin, and phenethyl resorcinol.

The moisturizing component includes at least one of glycerin and tetrahydromethylpyrimidine carboxylic acid.

The antioxidant component includes at least one of glucosyl hesperidin and vitamin C ethyl ether.

The eye bag removing component contains at least one of niacinamide and caffeine.

The anti-aging component contains at least one of tocopherol and retinol.

In other preferred embodiments of the present disclosure, the microneedle formulation contains drugs. The drugs include, but not limited to, GLP-1 analogues, parathyroid hormone analogues, GH, donepezil, triamcinolone acetonide, or doxorubicin.

In other preferred embodiments of the present disclosure, the microneedle formulation contains vaccines. The vaccines include, but not limited to, inactivated influenza vaccines, hepatitis B virus vaccines, SARS-CoV-2 vaccines, or mRNA vaccines.

The present disclosure also provides a preparation method for microneedle formulation. For water-soluble active components, the method includes the following steps: dissolving the aforementioned formulation of microneedle scaffold material, surfactant with cyclic structure, and active components in deionized water, stirring evenly, and degassing, to obtain the microneedle formulation.

For water-insoluble or poorly water-soluble active components, the method includes the following steps:

(1) Dissolving the formulation amount of microneedle scaffold material in a portion of deionized water, stir evenly to obtain a scaffold solution.

(2) Adding the formulation amount of surfactant with cyclic structure and active components to the remaining portion of deionized water, and stirring evenly to obtain an active solution.

(3) Mixing the scaffold solution obtained in step (1) with the active solution obtained in step (2), stirring evenly, and degassing, to obtain the microneedle formulation.

Preferably, the degassing process includes, but is not limited to, vacuum and/or centrifugation.

The present disclosure also provides a microneedle patch, which is made from the aforementioned microneedle formulation.

The present disclosure also provides a preparation method for the aforementioned microneedle patch, including the following steps:

S1. Infusing the microneedle formulation into the mold and dry the mold.

S2. Peeling off the microneedle from the dried mold obtained in step S1 to obtain a microneedle sheet.

S3. Attaching an adhesive tape to the back of the microneedle sheet obtained in step S2 to obtain the microneedle patch.

Preferably, the infusion in step S1 includes high-pressure infusion, centrifugation, vacuum adsorption, or self-leveling.

Preferably, the microneedle sheet in step S2 can be directly peeled off to form the target shape, or it can be cut into the target shape after peeling off.

The present disclosure also provides a microneedle patch containing a microneedle base and microneedle bodies located on the microneedle base. The microneedle body includes a microneedle root and a microneedle tip, and the microneedle root connects the microneedle base and the microneedle tip. Where, the microneedle root and/or the microneedle base is made from the aforementioned microneedle formulation without active components.

Preferably, the microneedle bodies contain active components, where the active components are drugs, vaccines, or skincare components.

The present disclosure also provides a method for preparing the aforementioned microneedle patch, including the following steps:

S1. Infusing a microneedle body formulation containing active components into a mold to form microneedle bodies or microneedle tips.

S2. Infusing a microneedle formulation without active components into the mold containing the microneedle bodies or the microneedle tips obtained in step S1 to form microneedle roots and/or a microneedle base.

S3. Drying and peeling the mold containing the microneedle roots and/or the microneedle base obtained in step S2 to obtain a microneedle sheet.

S4. Attaching an adhesive tape to the back of the microneedle sheet obtained in step S4 to obtain the microneedle patch.

Preferably, the microneedle formulation in step S1 also includes microneedle scaffold material.

Preferably, the infusion in steps S1 and S2 includes high-pressure infusion, centrifugation, vacuum adsorption, or self-leveling.

Preferably, before infusing the microneedle formulation without active components in step S2, the step to dry the mold may be included, or it may be omitted.

Preferably, the microneedle sheet in step S3 can be directly peeled off from the mold to form the target shape, or it can be cut to form the target shape after peeling off.

The beneficial effects of the present disclosure are as follows:

(1) The formulation of the microneedle preparation is improved by using a combination of large and small molecule microneedle scaffold material, which stabilizes the shaping of the microneedle and ensures the yield rate and user experience of the microneedle. The addition of surfactant with cyclic structure can enhance the stability of internal bubbles in the formulation, preventing them from aggregating and rupturing. This significantly increases the yield rate by avoiding the generation of defective products due to bubble aggregation during the entire drying process, addressing the issue of bubbles easily aggregating and rupturing in the production of microneedle using existing mold-based methods.

(2) The surfactant with cyclic structure used in the present disclosure has good compatibility with various microneedle scaffold material and a wide range of applications. Additionally, the surfactant with cyclic structure has emulsifying abilities, improving the solubility of raw material. This allows other materials that are insoluble or poorly soluble in water to achieve higher solubility and better stability, thereby increasing the yield rate of the product.

(3) Experimental results show that compared to other non-ionic surfactant, the combination of surfactant with cyclic structure and microneedle scaffold material can effectively prevent the curling of microneedle sheet surfaces, improving the flatness of the product and enhancing product quality. This may be an unexpected benefit brought by the unique spatial structure of surfactant with cyclic structure.

(4) The surfactant with cyclic structure used in the present disclosure include non-ionic surfactant with cyclic structure and plant-derived surfactant with cyclic structure, which have low irritation to the human body and high safety. They also have the effect of promoting blood microcirculation. When equipped with other functional components, they can accelerate the subcutaneous diffusion of functional components to the target site, enhance efficacy, and help improve skin color darkened by blood vessels.

(5) In the preparation method, insoluble or poorly soluble active components are mixed with surfactant with cyclic structure, and then mixed with the microneedle scaffold solution. The surfactant with cyclic structure can promote better solubility and stability of the insoluble or poorly soluble active components.

(6) In the present disclosure, the segmented preparation of microneedle patch concentrates the active component in the microneedle body, especially in the needle tip area, enabling precise control of the dosage of active component and better efficacy.

### BRIEF DESCRIPTION OF THE DRAWINGS

FIG. 1 illustrates flatness comparison of Embodiments 1-9 and Comparative Examples 1-6.

FIG. 2 illustrates dissolution comparison of soluble microneedle formulations for removing eye bags of Embodiment 24 and Comparative Example 9.

FIG. 3 illustrates dissolution comparison of anti-aging soluble microneedle formulations of Embodiment 25 and Comparative Example 11.

FIG. 4 illustrates comparison of needle deficiency rate of soluble microneedle patches for removing eye bags prepared in Embodiment 24 and Comparative Example 9.

FIG. 5 illustrates flatness comparison of soluble microneedle patches for removing eye bags prepared in Embodiment 24 and Comparative Example 10.

Where, S in the figures represents embodiment, and D represents comparative example.

### DETAILED DESCRIPTION

The embodiments of the present disclosure are described below with reference to specific examples. Those skilled in the art can easily understand other advantages and effects of the present disclosure from the content disclosed in this specification. The present disclosure can also be implemented or applied through other different specific embodiments. Various details in this specification can also be modified or changed in various ways based on different viewpoints and applications without departing from the spirit of the present disclosure.

Before further describing the specific embodiments of the present disclosure, it should be understood that the protection scope of the present disclosure is not limited to the following specific embodiments; it should also be understood that the terms used in the embodiments of the present disclosure are for describing specific embodiments, and not intended to limit the scope of the present disclosure.

The present disclosure does not limit the source of the raw material used. Unless otherwise specified, the raw material used in the present disclosure are all common commercial products in this technical field.

A microneedle formulation contains the following components in mass percentage: 15-55% microneedle scaffold material, 0.05-5% surfactant with cyclic structure, and the remaining being deionized water.

In some embodiments, the surfactant with cyclic structure includes at least one of a pyridinium salt, a morpholinium salt, an imidazolinium salt, a piperazinium salt, and a saponin.

Specifically, the pyridinium salt includes at least one of a pyridone ethanolamine salt, a pyridinium hydrochloride salt, and a 2,6-diaminopyridine sulfate salt; the morpholinium salt includes a soybean oil-based ethylmorpholinium ethyl sulfate salt and/or an isostearamido propylmorpholinium lactate salt; the piperazinium salt includes a hydroxyethyl piperazine ethane sulfonate and/or p-chlorophenyl piperazine hydrochloride salt; the imidazolinium salt includes an isostearyl ethylimidazolinium ethyl sulfate salt; and the saponin includes at least one of a soapberry saponin, a ginsenoside, an astragalus saponin, an aesculus saponin, a diosgenin, and a ruscogenin.

Where, the astragalus saponin includes, but is not limited to, astragalus saponin I, II, III, IV, V, VI, and VII.

The microneedle scaffold material includes at least one of sodium hyaluronate, polyvinylpyrrolidone, chondroitin sulfate, sodium hydroxyethyl cellulose, collagen, sucrose, trehalose, maltose, chitosan, and dextran.

Preferably, the microneedle scaffold material includes a mass ratio of 1:1-3:1 of large molecule microneedle scaffold material to small molecule microneedle scaffold material; or a mass ratio of 0:1-0.5:1 of large molecule microneedle scaffold material to small molecule microneedle scaffold material. Wherein, the molecular weight of the large molecule microneedle scaffold material is ≥50,000 Da, and the molecular weight of the small molecule microneedle scaffold material is <50,000 Da.

In some preferable embodiments, the microneedle formulation includes the following components in mass percentage: 15-50% of microneedle scaffold material, 1-5% of surfactant with cyclic structure, and the remaining being deionized water.

In other preferable embodiments, the microneedle formulation includes the following components in mass percentage: 20-27% of microneedle scaffold material, 1.54% of surfactant with cyclic structure, and the remaining being deionized water.

The disclosure also provides a method for preparing a microneedle formulation, including the following steps: dissolving the aforementioned amounts of microneedle scaffold material and surfactant with cyclic structure in deionized water, stirring evenly, and degassing, thereby obtaining the microneedle formulation, where the degassing includes but is not limited to vacuum and/or centrifugation.

The disclosure further provides a microneedle patch and a preparation method thereof, where the microneedle patch is prepared from the aforementioned microneedle formulation.

Specifically, the method for preparing the microneedle patch includes the following steps:

S1. Infusing the microneedle formulation into a mold, where the infusion includes high-pressure infusion, vacuum adsorption, centrifugation, or self-leveling.

S2. Drying the mold with the infused microneedle formulation.

S3. Peeling off the microneedle from the dried mold to obtain a microneedle sheet. The microneedle sheet can be directly peeled off the mold to form the target shape or cut into the target shape after being peeled off the mold.

S4. Attaching an adhesive tape to the back of the microneedle sheet to obtain the microneedle patch.

1. Result Evaluation of Microneedle Patch without Active Component

### Microneedle Patch: Embodiments 1-20 and Comparative Examples 1-8

The formulations ofthe microneedle patch for Embodiments 1-20 and Comparative Examples 1-8 are shown in Table 1.

TABLE 1
Formulations of Microneedle Patch for Embodiments
and Comparative Examples (Unit: Parts by Weight)

| | | Mass | | | | |
|---|---|---|---|---|---|---|
| | | ratio of | | | | |
| | | large | | | | |
| | Microneedle | molecule | | | | Deionized |
| | scaffold | to small | Total | Surfactant with | | water/ |
| Group | material | molecule | portion | cyclic structure | portion | portion |
| Embodiment 1 | Large molecule | 1:1 | 15 | Pyridinium salt | 1 | 84 |
| | sodium hydroxyethyl | | | | | |
| | cellulose 7.5 parts | | | | | |
| | Small molecule | | | | | |
| | sodium hydroxyethyl | | | | | |
| | cellulose 7.5 parts | | | | | |
| Embodiment 2 | Large molecule | 3:1 | 24 | Morpholinium | 2 | 74 |
| | chondroitin sulfate18 | | | salt | | |
| | parts | | | | | |
| | Small molecule | | | | | |
| | chondroitin sulfate 6 | | | | | |
| | parts | | | | | |
| Embodiment 3 | Large molecule | 1:3 | 36 | Ruscogenin | 3 | 61 |
| | sodium hyaluronate 9 | | | | | |
| | parts | | | | | |
| | Small molecule | | | | | |
| | sodium hyaluronate 27 | | | | | |
| | parts | | | | | |
| Embodiment 4 | Large molecule | 2:7 | 45 | Piperazinium salt | 4 | 51 |
| | polyvinylpyrrolidone | | | | | |
| | 10 parts | | | | | |
| | Small molecule | | | | | |
| | polyvinylpyrrolidone | | | | | |
| | 35 parts | | | | | |
| Embodiment 5 | Large molecule | 0.36:1 | 55 | Imidazolinium | 5 | 40 |
| | sodium hyaluronate | | | salt | | |
| | 14.5 parts | | | | | |
| | Small molecule | | | | | |
| | sodium hyaluronate | | | | | |
| | 40.5 parts | | | | | |
| Embodiment 6 | Large molecule | 2.4:1 | 20 | Aesculus | 0.05 | 79.95 |
| | sodium hyaluronate | | | saponin | | |
| | 14.1 parts | | | | | |
| | Small molecule | | | | | |
| | sodium hyaluronate | | | | | |
| | 5.9 parts | | | | | |
| Embodiment 7 | Large molecule | 2:1 | 21 | Astragalus | 1.5 | 77.5 |
| | sodium hyaluronate 14 | | | saponin | | |
| | parts | | | | | |
| | Small molecule | | | | | |
| | sodium hyaluronate 7 | | | | | |
| | parts | | | | | |
| Embodiment 8 | Large molecule | 2:1 | 24 | Diosgenin | 2.5 | 73.5 |
| | sodium hyaluronate 16 | | | | | |
| | parts | | | | | |
| | Small molecule | | | | | |
| | sodium hyaluronate 8 | | | | | |
| | parts | | | | | |
| Embodiment 9 | Large molecule | 2.8:1 | 27 | Soapberry | 4 | 69 |
| | sodium hyaluronate | | | saponin | | |
| | 19.9 parts | | | | | |
| | Small molecule | | | | | |
| | sodium hyaluronate | | | | | |
| | 7.1 parts | | | | | |
| Embodiment 10 | Small molecule | 0:1 | 50 | Astragalus | 0.5 | 49.5 |
| | sodium hyaluronate 50 | | | saponin | | |
| | parts | | | | | |
| Embodiment 11 | Large molecule | 3:7 | 50 | Pyridinium salt | 1 | 49 |
| | sodium hyaluronate 15 | | | | | |
| | parts | | | | | |
| | Small molecules: 20 | | | | | |
| | parts of sucrose, 15 | | | | | |
| | parts of | | | | | |
| | polyvinylpyrrolidone | | | | | |
| Embodiment 12 | Small molecule | 0:1 | 15 | Aesculus | 0.1 | 84.9 |
| | polyvinylpyrrolidone | | | saponin | | |
| | 15 parts | | | | | |
| Embodiment 13 | Large molecule | 0.36:1 | 34 | Pyridinium salt | 3 | 63 |
| | polyvinylpyrrolidone | | | | | |
| | 9 parts | | | | | |
| | Small molecule | | | | | |
| | sodium hyaluronate 25 | | | | | |
| | parts | | | | | |
| Embodiment 14 | Large molecule | 1.5:1 | 15 | Morpholinium | 3 | 82 |
| | sodium hyaluronate 9 | | | salt | | |
| | parts | | | | | |
| | Small molecule | | | | | |
| | trehalose 6 parts | | | | | |
| Embodiment 15 | Large molecule | 0.2:1 | 30 | Ginsenoside | 5 | 65 |
| | polyvinylpyrrolidone | | | | | |
| | 5 parts | | | | | |
| | Small molecule | | | | | |
| | sodium hyaluronate 25 | | | | | |
| | parts | | | | | |
| Embodiment 16 | Large molecule | 0.5:1 | 30 | Ginsenoside | 5 | 65 |
| | polyvinylpyrrolidone | | | | | |
| | 10 parts | | | | | |
| | Small molecules: 18 | | | | | |
| | parts of sodium | | | | | |
| | hyaluronate, 2 parts of | | | | | |
| | maltose | | | | | |
| Embodiment 17 | Large molecule | 0.22:1 | 28 | Morpholinium | 2 | 70 |
| | hydroxyethyl cellulose | | | salt | | |
| | 5 parts | | | | | |
| | Small molecules: 20 | | | | | |
| | parts of sodium | | | | | |
| | hyaluronate, 3 parts of | | | | | |
| | trehalose | | | | | |
| Embodiment 18 | Large molecule | 0.33:1 | 40 | Pyridinium salt | 3 | 57 |
| | polyvinylpyrrolidone | | | | | |
| | 10 parts | | | | | |
| | Small molecules: 10 | | | | | |
| | parts of sodium | | | | | |
| | hyaluronate, 10 parts | | | | | |
| | of collagen, 10 parts | | | | | |
| | of sodium | | | | | |
| | hydroxyethyl cellulose | | | | | |
| Embodiment 19 | Large molecule | 0.5:1 | 45 | Ginsenoside | 5 | 50 |
| | polyvinylpyrrolidone | | | | | |
| | 15 parts | | | | | |
| | Small molecules: 10 | | | | | |
| | parts of sodium | | | | | |
| | hyaluronate, 20 parts | | | | | |
| | of chitosan | | | | | |
| Embodiment 20 | Large molecule | 0.32:1 | 33 | Morpholinium | 4 | 63 |
| | sodium hyaluronate 8 | | | salt | | |
| | parts | | | | | |
| | Small molecules: 5 | | | | | |
| | parts of sodium | | | | | |
| | hyaluronate, 10 parts | | | | | |
| | of dextran, 5 parts of | | | | | |
| | sucrose, 5 parts of | | | | | |
| | collagen | | | | | |
| Comparative | Large molecule | 1:2 | 58 | Astragalus | 2 | 40 |
| Example 1 | sodium hyaluronate | | | saponin | | |
| | 19.3 parts | | | | | |
| | Small molecule | | | | | |
| | sodium hyaluronate | | | | | |
| | 38.7 parts | | | | | |
| Comparative | Large molecule | 2:1 | 21 | Astragalus | 6 | 73 |
| Example 2 | sodium hyaluronate 14 | | | saponin | | |
| | parts | | | | | |
| | Small molecule | | | | | |
| | sodium hyaluronate 7 | | | | | |
| | parts | | | | | |
| Comparative | Large molecule | 2:1 | 24 | Deionized water | 2.5 | 73.5 |
| Example 3 | sodium hyaluronate 16 | | | | | |
| | parts | | | | | |
| | Small molecule | | | | | |
| | sodium hyaluronate 8 | | | | | |
| | parts | | | | | |
| Comparative | Large molecule | 2:1 | 24 | Twain-20 | 2.5 | 73.5 |
| Example 4 | sodium hyaluronate 16 | | | | | |
| | parts | | | | | |
| | Small molecule | | | | | |
| | sodium hyaluronate 8 | | | | | |
| | parts | | | | | |
| Comparative | Large molecule | 2:1 | 24 | Hydrogenated | 2.5 | 73.5 |
| Example 5 | sodium hyaluronate 16 | | | castor oil | | |
| | parts | | | | | |
| | Small molecule | | | | | |
| | sodium hyaluronate 8 | | | | | |
| | parts | | | | | |
| Comparative | Large molecule | 2:1 | 24 | Fatty Acyl | 2.5 | 73.5 |
| Example 6 | sodium hyaluronate 16 | | | Diethanolamine | | |
| | parts | | | | | |
| | Small molecule | | | | | |
| | sodium hyaluronate 8 | | | | | |
| | parts | | | | | |
| Comparative | Large molecule | 0.8:1 | 27 | Soapberry | 4 | 69 |
| Example 7 | sodium hyaluronate 12 | | | saponin | | |
| | parts | | | | | |
| | Small molecule | | | | | |
| | sodium hyaluronate 15 | | | | | |
| | parts | | | | | |
| Comparative | Large molecule | 3.2:1 | 27 | Soapberry | 4 | 69 |
| Example 8 | sodium hyaluronate | | | saponin | | |
| | 20.6 parts | | | | | |
| | Small molecule | | | | | |
| | sodium hyaluronate | | | | | |
| | 6.4 parts | | | | | |

The microneedle scaffold material is a mixture of large molecule microneedle scaffold material and small molecule microneedle scaffold material, where the molecular weight of the large molecule microneedle scaffold material is ≥50,000 Da, and the molecular weight of the small molecule microneedle scaffold material is <50,000 Da.

The method for preparing the microneedle patch formulation includes the following steps: thoroughly mixing and dissolving the microneedle scaffold material and the surfactant with cyclic structure in deionized water, placing the mixture in a centrifugal mixing device, and processing for 30 minutes to obtain the microneedle formulation.

The method for preparing the microneedle patch includes the following steps:

(1) Infusing the microneedle formulation into a mold using a high-pressure infusion method.

(2) Placing the mold containing the microneedle formulation in a drying box for drying.

(3) Removing the mold from the drying box and peeling off an entire microneedle sheet from the mold. If the peeled microneedle sheet is not in the target shape, it can be cut to form the microneedle sheet in the target shape.

(4) Attaching an adhesive tape to the back of the microneedle sheet to obtain the microneedle patch.

(5) Packaging and sterilization.

The results of the microneedle patch prepared in Embodiments 1-20 and Comparative Examples 1-8 in terms of needle deficiency rate, unflatness rate, yield rate, etc. are shown in Table 2.

TABLE 2
Quality of microneedle patch.

| | Needle deficiency | | |
|---|---|---|---|
| | rate % | Unflatness rate % | Yield rate % |
| Embodiment 1 | 15 | 2.1 | 82.9 |
| Embodiment 2 | 12.5 | 0 | 87.5 |
| Embodiment 3 | 8.6 | 1.3 | 90.1 |
| Embodiment 4 | 7.5 | 0.8 | 91.7 |
| Embodiment 5 | 10.2 | 1.6 | 88.2 |
| Embodiment 6 | 8.0 | 0 | 92.0 |
| Embodiment 7 | 7.3 | 0 | 92.7 |
| Embodiment 8 | 5.4 | 0 | 94.6 |
| Embodiment 9 | 6.2 | 0 | 93.8 |
| Embodiment 10 | 0 | 7.4 | 92.6 |
| Embodiment 11 | 8.6 | 0 | 91.4 |
| Embodiment 12 | 2 | 3 | 95 |
| Embodiment 13 | 2.4 | 8.4 | 89.2 |
| Embodiment 14 | 3.9 | 5.7 | 90.4 |
| Embodiment 15 | 4.6 | 4.8 | 90.6 |
| Embodiment 16 | 0 | 6.5 | 93.5 |
| Embodiment 17 | 1.7 | 7.3 | 91 |
| Embodiment 18 | 5 | 4.6 | 90.4 |
| Embodiment 19 | 4.8 | 6.7 | 88.5 |
| Embodiment 20 | 7.5 | 3.2 | 89.3 |
| Comparative | 6.0 | 32.8 | 61.2 |
| Example 1 | | | |
| Comparative | 6.3 | 70.8 | 22.9 |
| Example 2 | | | |
| Comparative | 64.6 | 15.4 | 20 |
| Example 3 | | | |
| Comparative | 10.8 | 61.3 | 27.9 |
| Example 4 | | | |
| Comparative | 9.1 | 45.8 | 45.1 |
| Example 5 | | | |
| Comparative | 5.8 | 52.1 | 42.1 |
| Example 6 | | | |
| Comparative | 34 | 31.5 | 34.5 |
| Example 7 | | | |
| Comparative | 31.2 | 41.2 | 27.6 |
| Example 8 | | | |

Based on the results of Embodiments 1-20 in the above table, it can be seen that the microneedle patch made from microneedle scaffold material and surfactants with cyclic structure, as disclosed in this disclosure, can effectively reduce the needle deficiency rate and unflatness rate, thereby significantly improving the yield rate of microneedle patch production.

Furthermore, through Embodiment 7 and Comparative Examples 1 and 2, it is observed that significant increases in the unflatness rate of microneedle patch occur when the quantities of microneedle scaffold material or surfactant are altered beyond the scope of protection specified in this disclosure.

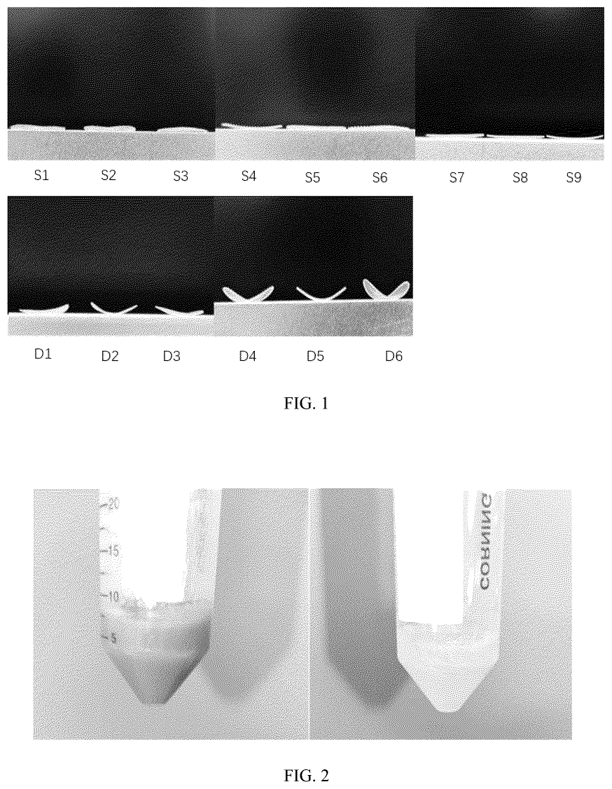

From Embodiment 8 and Comparative Examples 3-6, it is noted that substituting deionized water for surfactant has a significant impact on both the needle deficiency rate and unflatness rate of microneedle patch prepared in Comparative Example 3. When using other conventional surfactants, as shown in Comparative Examples 4-6, although the needle deficiency rate is minimally affected, there is a notable increase in the unflatness rate of the prepared microneedle patch, as depicted in FIG. 1. The above findings indicate that conventional surfactants, when combined with microneedle scaffold material, do not consistently improve the flatness rate of microneedle patch product. Only the specific surfactants with cyclic structures as disclosed in this disclosure can achieve the aforementioned technical effects. This may be attributed to the unique spatial structure of surfactants with cyclic structures, which bring about unexpected benefits.

Through Embodiment 9 and Comparison Examples 7 and 8, it can be seen that altering the mass ratio of large molecule scaffold material to small molecule scaffold material within the microneedle scaffold outside the scope of the present disclosure also fails to achieve the desired technical effect of reducing the needle deficiency rate and unflatness rate of microneedle patch.

In summary, the present disclosure improves the microneedle formulation by including both large molecule scaffold material and small molecule scaffold material to enhance the stability of microneedle shaping. The addition of surfactants with cyclic structure enhances the stability of bubbles within the formulation, preventing aggregation and rupturing, thereby significantly increasing the yield rate. This addresses the issue of bubbles easily aggregating and rupturing during the production of microneedles using existing mold methods.

In other preferable embodiments of the present disclosure, the microneedle formulation may also include a mass percentage of no more than 15% of active component. The active component includes, but is not limited to, drugs, vaccines, or skincare components.

In some preferable technical solutions, the skincare component includes, but is not limited to, a whitening component, a moisturizing component, an antioxidant component, an eye bag removing component, an anti-aging component, and a vascular color reducing component.

Specifically, the whitening component includes at least one of glabridin, niacinamide, phloretin, and phenethyl resorcinol.

The moisturizing component includes at least one of glycerin and tetrahydromethylpyrimidine carboxylic acid.

The antioxidant component includes at least one of glucosyl hesperidin and vitamin C ethyl ether.

The eye bag removing component includes at least one of niacinamide and caffeine.

The anti-aging component includes at least one of tocopherol and retinol.

In some preferable technical solutions, the drug includes, but is not limited to, GLP-1 analogues, parathyroid hormone analogues, GH, donepezil, triamcinolone acetonide, or doxorubicin.

In some technical solutions, the vaccine includes, but is not limited to, an inactivated influenza vaccine, a hepatitis B virus vaccine, a SARS-CoV-2 vaccine, or an mRNA vaccine.

The present disclosure further provides a method for preparing a microneedle formulation, which includes the following steps for water-soluble active components: dissolving a formulation amount of microneedle scaffold material, a surfactant with a cyclic structure, and an active component in deionized water, stirring evenly, and degassing to obtain the microneedle formulation.

For active components that are poorly soluble or insoluble in water, the method includes the following steps:

(1) Dissolving a formulation amount of microneedle scaffold material in a portion of deionized water, and stirring evenly to obtain a scaffold solution.

(2) Adding a formulation amount of surfactant with a cyclic structure and an active component to the remaining portion of deionized water, and stirring evenly to obtain an active solution.

(3) Mixing the scaffold solution obtained in step (1) with the active solution obtained in step (2), stirring evenly, and degassing to obtain the microneedle formulation.

Preferably, the degassing process includes, but is not limited to, vacuum and/or centrifugation.

The present disclosure also provides a microneedle patch prepared from the aforementioned microneedle formulation.

The present disclosure also provides a method for preparing the aforementioned microneedle patch, including the following steps:

S1. Infusing the microneedle formulation into a mold, where the infusion includes high-pressure infusion, centrifugation, vacuum adsorption, or self-leveling.

S2. Drying the mold after infusion of the microneedle formulation.

S3. Peeling off the microneedles from the dried mold to obtain a microneedle sheet. The microneedle sheet can be directly peeled off from the mold to form the target shape, or can be cut into the target shape after peeling off from the mold.

S4. Attaching an adhesive tape to the back of the microneedle sheet, thereby obtaining the microneedle patch.

The present disclosure further provides a microneedle patch containing active components, including a microneedle base and microneedle bodies located on the microneedle base. The microneedle body includes a microneedle root and a microneedle tip, with the microneedle root connecting the microneedle base and the microneedle tip. The microneedle bodies or microneedle tips are made from a microneedle body formulation containing active components and microneedle scaffold material. The microneedle root and/or microneedle base are made from the aforementioned microneedle formulation without active components. The active component includes a drug, a vaccine, or a skincare component. Specifically, the drug includes GLP-1 analogues, parathyroid hormone analogues, GH, triamcinolone acetonide, or doxorubicin. The vaccine includes inactivated influenza vaccines, hepatitis B virus vaccines, SARS-COV-2 vaccines, or mRNA vaccines. The skincare component includes, but is not limited to, a whitening component, a moisturizing component, an antioxidant component, an eye bag removing component, an anti-aging component, and a vascular color reducing component.

The present disclosure further provides a method for preparing the aforementioned microneedle patch, including the following steps:

S1. Infusing a microneedle body formulation containing active components into a mold to form microneedle bodies or microneedle tips.

S2. Infusing a microneedle formulation without active components into the mold containing microneedle bodies or microneedle tips from step S1 to form microneedle roots and/or a microneedle base.

S3. Drying and demolding the mold containing the microneedle roots and/or the microneedle base from step S2 to obtain a microneedle sheet.

S4. Attaching an adhesive tape to the back of the microneedle sheet obtained in step S4 to obtain the microneedle patch.

Preferably, the microneedle body formulation in step S1 further includes microneedle scaffold material.

Preferably, before infusing the microneedle formulation without active components in step S2, a drying step of the mold may be included, or the drying step of the mold may be omitted.

Preferably, the microneedle formulation in step S2 includes the microneedle scaffold material, surfactant with a cyclic structure, and deionized water.

Preferably, the infusion in steps S1 and S2 includes high-pressure infusion, vacuum adsorption, centrifugation, or self-leveling.

Preferably, the microneedle sheet in step S3 can be directly demolded to form the target shape, or it can be cut to form the target shape after demolding.

### Embodiment 21: Whitening Soluble Microneedle Formulation and Microneedle Patch

The whitening soluble microneedle formulation, in parts by weight, includes the following components: 22 parts of sodium hyaluronate, 1 part of glabridin, 3 parts of niacinamide, 2 parts of phloretin, 1 part of phenethyl resorcinol, 2 parts of soapberry saponin, and 69 parts of deionized water.

The sodium hyaluronate is a mixture of large molecule sodium hyaluronate and small molecule sodium hyaluronate, where the molecular weight of the large molecule sodium hyaluronate is ≥50,000 Da, and the molecular weight of the small molecule sodium hyaluronate is <50,000 Da, and the mass ratio of large molecule sodium hyaluronate to small molecule sodium hyaluronate is 3:1.

The preparation method of the whitening soluble microneedle formulation in this embodiment includes the following steps:

(1) Stirring and dissolving 49 parts of deionized water and 22 parts of sodium hyaluronate thoroughly to obtain solution A.

(2) Adding glabridin, niacinamide, phloretin, phenethyl resorcinol, and soapberry saponin to 20 parts of deionized water, stirring and dissolving thoroughly to obtain solution B.

(3) Mixing and stirring solution A and solution B thoroughly to obtain the whitening soluble microneedle formulation.

The preparation method of the whitening soluble microneedle patch in this embodiment includes the following steps:

(1) Infusing the microneedle formulation into a mold using a high-pressure infusion method.

(2) Placing the mold in a drying box for drying.

(3) Removing the mold from the drying box and demolding an entire microneedle sheet.

(4) Attaching an adhesive tape to the back of the microneedle sheet to obtain the microneedle patch.

(5) Packaging and sterilization.

### Embodiment 22: Moisturizing Soluble Microneedle Formulation and Microneedle Patch

The moisturizing soluble microneedle formulation, in parts by weight, includes the following components: 15 parts of sodium hydroxyethyl cellulose, 7.5 parts of glycerin, 7.5 parts of tetrahydromethylpyrimidine carboxylic acid, 1 part of ginsenoside, and 69 parts of deionized water.

Where, sodium hydroxyethyl cellulose is a mixture of large molecule sodium hydroxyethyl cellulose and small molecule sodium hydroxyethyl cellulose. The molecular weight of large molecule sodium hydroxyethyl cellulose is ≥50,000 Da, and the molecular weight of small molecule sodium hydroxyethyl cellulose is <50,000 Da. The mass ratio of large molecule sodium hydroxyethyl cellulose to small molecule sodium hydroxyethyl cellulose is 1:1.

The preparation method of the moisturizing soluble microneedle formulation in this embodiment includes the following steps:

Mixing and stirring 69 parts of deionized water with sodium hydroxyethyl cellulose, glycerin, Tetrahydromethylpyrimidine carboxylic acid, and ginsenoside thoroughly, and then placing the mixture in a centrifugal mixing device and processing for 30 minutes to obtain the moisturizing soluble microneedle formulation.

The preparation method of the moisturizing soluble microneedle patch in this embodiment includes the following steps:

(1) Infusing the microneedle formulation into a mold using the centrifugation method.

(2) Placing the mold in a drying box for drying.

(3) Removing the mold from the drying box and demolding the entire microneedle sheet.

(4) Attaching an adhesive tape to the back of the microneedle sheet, to obtain the microneedle patch.

(5) Packaging and sterilization.

### Embodiment 23: Antioxidant Soluble Microneedle Formulation and Microneedle Patch

The antioxidant soluble microneedle formulation, in parts by weight, includes the following components: 45 parts of sodium hydroxyethyl cellulose, 2 parts of glucosyl hesperidin, 4 parts of vitamin C ethyl ether, 2 parts of astragalus saponin and 47 parts of deionized water.

Where, sodium hydroxyethyl cellulose is a mixture of large molecule sodium hydroxyethyl cellulose and small molecule sodium hydroxyethyl cellulose. The molecular weight of large molecule sodium hydroxyethyl cellulose is ≥50,000 Da, and the molecular weight of small molecule sodium hydroxyethyl cellulose is <50,000 Da. The mass ratio of large molecule sodium hydroxyethyl cellulose to small molecule sodium hydroxyethyl cellulose is 2:1.

The preparation method of the antioxidant soluble microneedle formulation in this embodiment includes the following steps:

Mixing and stirring 47 parts of deionized water with sodium hydroxyethyl cellulose, glucosyl hesperidin, vitamin C ethyl ether, and astragalus saponin, and then placing the mixture in a centrifugal mixing device and processing for 30 minutes, to obtain the antioxidant soluble microneedle patch formulation.

The preparation method of the antioxidant soluble microneedle patch in this embodiment includes the following steps:

(1) Infusing the microneedle formulation into a mold using a leveling method.

(2) Placing the mold in a drying box for drying.

(3) Removing the mold from the drying box and demolding the entire microneedle sheet.

(4) Attaching an adhesive tape to the back of the microneedle sheet, to obtain the microneedle patch.

(5) Packaging and sterilization.

### Embodiment 24: Soluble Microneedle Formulation and Microneedle Patch for Eye Bag Removing

The soluble microneedle formulation for eye bag removing, in parts by weight, includes the following components: 24 parts of sodium hyaluronate, 0.5 parts of niacinamide, 0.5 parts of caffeine, 1 part of aesculus saponin, and 74 parts of deionized water.

Where, the sodium hyaluronate is a mixture of large molecule sodium hyaluronate and small molecule sodium hyaluronate. The molecular weight of large molecule sodium hyaluronate is ≥50,000 Da, and the molecular weight of small molecule sodium hyaluronate is <50,000 Da. The mass ratio of large molecule sodium hyaluronate to small molecule sodium hyaluronate is 2.4:1.

The preparation method of the soluble microneedle formulation for eye bag removing includes the following steps:

(1) Mixing and stirring thoroughly 44 parts of deionized water with 24 parts of sodium hyaluronate to obtain solution A.

(2) Dissolving niacinamide, caffeine, and aesculus saponin into 30 parts of deionized water, and mixing well to obtain solution B.

(3) Mixing solution A and solution B thoroughly to obtain the target microneedle formulation.

The preparation method of the soluble microneedle patch for eye bag removing includes the following steps:

(1) Infusing the microneedle formulation into the mold using high-pressure infusing.

(2) Placing the mold in a drying box for drying.

(3) Removing the mold from the drying box and peeling off an entire microneedle sheet from the mold.

(4) Attaching an adhesive tape to the back of the microneedle sheet to obtain the microneedle patch.

(5) Packaging and sterilization.

### Embodiment 25: Anti-Aging Soluble Microneedle Formulation and Microneedle Patch

The anti-aging soluble microneedle formulation, in parts by weight, includes the following components: 28 parts of sodium hyaluronate, 1 part of tocopherol, 1 part of retinol, 4 parts of diosgenin, and 66 parts of deionized water.

Where, the sodium hyaluronate is a mixture of large molecule sodium hyaluronate and small molecule sodium hyaluronate. The molecular weight of large molecule sodium hyaluronate is ≥50,000 Da, and the molecular weight of small molecule sodium hyaluronate is <50,000 Da. The mass ratio of large molecule sodium hyaluronate to small molecule sodium hyaluronate is 2.8:1.

The preparation method of the anti-aging soluble microneedle formulation includes the following steps:

(1) Mixing thoroughly 56 parts of deionized water with 28 parts of sodium hyaluronate to obtain solution A.

(2) Dissolving tocopherol, retinol, and diosgenin in 10 parts of deionized water, and mixing well to obtain solution B.

(3) Mixing solution A and solution B thoroughly to obtain the anti-aging soluble microneedle formulation.

The preparation method of the anti-aging soluble microneedle patch includes the following steps:

(1) Infusing the microneedle formulation into the mold using high-pressure infusion.

(2) Placing the mold in a drying box for drying.

(3) Removing the mold from the drying box and peeling off an entire microneedle sheet from the mold.

(4) Attaching an adhesive tape to the back of the microneedle sheet to obtain the microneedle patch.

(5) Packaging and sterilization.

### Embodiment 26: Triamcinolone Acetonide-Containing Soluble Microneedle Formulation and Microneedle Patch

The microneedle body includes a soluble microneedle formulation containing triamcinolone acetonide, which includes the following components in parts by weight: 3 parts of small molecule sodium hyaluronate, 10 parts of triamcinolone acetonide, and 87 parts of deionized water.

The microneedle roots and/or the microneedle base are composed of a soluble microneedle formulation with 0% content of active component, which includes the following components in parts by weight: 50 parts of small molecule sodium hyaluronate, 1 part of astragalus saponin, and 49 parts of deionized water.

Where, the molecular weight of the small molecule sodium hyaluronate is <50,000 Da.

The preparation method of the soluble microneedle formulation containing triamcinolone acetonide includes the following steps:

(1) Preparation of the microneedle body formulation: mixing thoroughly 3 parts of small molecule sodium hyaluronate, 10 parts of triamcinolone acetonide, and 87 parts of deionized water to obtain solution A.

(2) Preparation of the microneedle formulation: mixing thoroughly 50 parts of small molecule sodium hyaluronate, 1 part of astragalus saponin, and 49 parts of deionized water to obtain solution B.

The preparation method of the microneedle patch containing triamcinolone acetonide includes the following steps:

(1) Infusing solution A containing triamcinolone acetonide into the mold using high-pressure infusion to form the microneedle bodies or the microneedle tips with active components.

(2) After drying the mold containing the microneedle bodies or the microneedle tips from step (1), using a leveling method to infuse solution B into the mold to form microneedle roots and/or a microneedle base without active components.

(3) Placing the mold containing the microneedle roots and/or the microneedle base from step (2) in a drying box for drying.

(4) Removing the mold from the drying box and peeling off an entire microneedle sheet from the mold.

(5) Attaching an adhesive tape to the back of the microneedle sheet to obtain the microneedle patch.

(6) Packaging and sterilization.

### Embodiment 27: Soluble Microneedle Formulation and Microneedle Patch Containing GLP-1 Analogues

The microneedle body includes a soluble microneedle formulation containing GLP-1 analogues, including the following components in parts by weight: 3 parts of small molecule sodium hyaluronate, 3 parts of GLP-1 analogues, and 94 parts of deionized water.

The microneedle roots and/or the microneedle base are composed of the soluble microneedle formulation with 0% content of active component, including the following components in parts by weight: 15 parts of large molecule sodium hyaluronate, 20 parts of small molecule sucrose, 15 parts of small molecule polyvinylpyrrolidone, 1 part of pyridinium salt, and 49 parts of deionized water.

Where, the molecular weight of large molecule sodium hyaluronate is ≥50,000 Da, and the molecular weights of small molecule polyvinylpyrrolidone and sucrose are both <50,000 Da.

In this embodiment, the preparation method of the soluble microneedle formulation containing the GLP-1 analogues includes the following steps:

(1) Preparation of the microneedle body formulation: mixing and dissolving 3 parts of small molecule sodium hyaluronate, 3 parts of GLP-1 analogues, and 94 parts of deionized water, to form solution A.

(2) Preparation of the microneedle formulation: mixing and dissolving 15 parts of large molecule sodium hyaluronate, 20 parts of small molecule sucrose, 15 parts of small molecule polyvinylpyrrolidone, 1 part of pyridinium salt, and 49 parts of deionized water, to form solution B.

The preparation method of the microneedle patch containing the GLP-1 analogues in this embodiment includes the following steps:

(1) Using high-pressure infusion to infuse solution A containing the GLP-1 analogues into a mold to form microneedle bodies or microneedle tips with active components.

(2) Using a leveling method to infuse solution B into the mold containing the microneedle bodies or the microneedle tips from step (1) to form microneedle roots and/or a microneedle base without active components.

(3) Placing the mold containing the microneedle roots and/or the microneedle base from step (2) in a drying box for drying.

(4) After removing the mold from the drying box, peeling off an entire microneedle sheet from the mold.

(5) Attaching an adhesive tape to the back of the microneedle sheet, to obtain the microneedle patch.

(6) Packaging and sterilization.

### Embodiment 28: Doxorubicin-Containing Soluble Microneedle Formulation and Microneedle Patch

The microneedle body includes a soluble microneedle formulation containing doxorubicin, including the following components in parts by weight: 3 parts of small molecule sodium hyaluronate, 10 parts of doxorubicin, and 87 parts of deionized water.

The microneedle roots and the microneedle base are composed of the soluble microneedle formulation with 0% content of active component, including the following components in parts by weight: 15 parts of small molecule polyvinylpyrrolidone, 5 parts of aesculus saponin and 80 parts of deionized water.

Where, the molecular weight of small molecule polyvinylpyrrolidone is <50,000 Da.

In this embodiment, the preparation method of the soluble microneedle formulation containing the doxorubicin includes the following steps:

(1) Preparation of the microneedle body formulation: mixing and dissolving 3 parts of small molecule sodium hyaluronate, 10 parts of doxorubicin, and 87 parts of deionized water, to form solution A.

(2) Preparation of the microneedle formulation: mixing and dissolving 15 parts of small molecule polyvinylpyrrolidone, 5 parts of aesculus saponin and 80 parts of deionized water, to form solution B.

The preparation method of the microneedle patch containing doxorubicin in this embodiment includes the following steps:

(1) Using high-pressure infusion to infuse solution A containing doxorubicin into a mold to form the microneedle bodies or the microneedle tips with active components.

(2) Using a leveling method to infuse solution B into the mold containing the microneedle bodies or the microneedle tips from step (1) to form the microneedle roots and the microneedle base without active components.

(3) Placing the mold containing the microneedle roots and the microneedle base from step (2) in a drying box for drying.

(4) After removing the mold from the drying box, peeling off an entire microneedle sheet from the mold.

(5) Attaching an adhesive tape to the back of the microneedle sheet, to obtain the microneedle patch.

(6) Packaging and sterilization.

### Embodiment 29: Donepezil-Containing Soluble Microneedle Formulation and Microneedle Patch

The microneedle body includes a soluble microneedle formulation containing donepezil, including the following components in parts by weight: 4 parts of donepezil, 3 parts of small molecule sodium hyaluronate, and 93 parts of deionized water.

The microneedle roots and the microneedle base are composed of the soluble microneedle formulation with 0% content of active component, including the following components in parts by weight: 9 parts of large molecule polyvinylpyrrolidone, 25 parts of small molecule sodium hyaluronate, 3 parts of pyridinium salt, and 63 parts of deionized water.

Where, the molecular weight of large molecule polyvinylpyrrolidone is ≥50,000 Da, and the molecular weight of small molecule sodium hyaluronate is <50,000 Da.

In this embodiment, the preparation method of the soluble microneedle formulation containing the donepezil includes the following steps:

(1) Preparation of the microneedle body formulation: mixing 4 parts of donepezil, 3 parts of small molecule sodium hyaluronate, and 93 parts of deionized water, stirring and dissolving evenly to form solution A.

(2) Preparation of the microneedle formulation: mixing 9 parts of large molecule polyvinylpyrrolidone, 25 parts of small molecule sodium hyaluronate, 3 parts of pyridinium salt, and 63 parts of deionized water, stirring and dissolving evenly to form solution B.

The preparation method of the microneedle patch containing donepezil in this embodiment includes the following steps:

(1) Using high-pressure infusion to infuse solution A containing donepezil into a mold to form the microneedle bodies or the microneedle tips with active components.

(2) Using a leveling method to infuse solution B into the mold containing the microneedle bodies or the microneedle tips from step (1) to form the microneedle roots and the microneedle base without active components.

(3) Placing the mold containing the microneedle roots and the microneedle base from step (2) in a drying box for drying.

(4) After removing the mold from the drying box, peeling off an entire microneedle sheet from the mold.

(5) Attaching an adhesive tape to the back of the microneedle sheet, to obtain the microneedle patch.

(6) Packaging and sterilization.

### Embodiment 30: Soluble Microneedle Formulation and Microneedle Patch Containing Parathyroid Hormone Analogues

The microneedle body includes a soluble microneedle formulation containing parathyroid hormone analogues, including the following components in parts by weight: 5 parts of parathyroid hormone analogues, 3 parts of small molecule sodium hyaluronate, and 92 parts of deionized water.

The microneedle roots and the microneedle base are composed of the soluble microneedle formulation with 0% content of active component, including the following components in parts by weight: 7.5 parts of large molecule sodium hyaluronate, 7.5 parts of small molecule trehalose, 3 parts of morpholinium salt and 82 parts of deionized water.

Where, the molecular weight of large molecule sodium hyaluronate is ≥50,000 Da, and the molecular weight of small molecule trehalose is <50,000 Da.

In this embodiment, the preparation method of the soluble microneedle formulation containing parathyroid hormone analogues includes the following steps:

(1) Preparation of the microneedle body formulation: mixing 5 parts of parathyroid hormone analogues, 3 parts of small molecule sodium hyaluronate, and 92 parts of deionized water, stirring and dissolving evenly to form solution A.

(2) Preparation of the microneedle formulation: mixing 7.5 parts of large molecule sodium hyaluronate, 7.5 parts of small molecule trehalose, 3 parts of morpholinium salt, and 82 parts of deionized water, stirring and dissolving evenly to form solution B.

The preparation method of the microneedle patch containing parathyroid hormone analogues in this embodiment includes the following steps:

(1) Using high-pressure infusion to infuse solution A containing parathyroid hormone analogues into a mold to form the microneedle bodies or the microneedle tips with active components.

(2) Using a leveling method to infuse solution B into the mold containing the microneedle bodies or the microneedle tips from step (1) to form the microneedle roots and the microneedle base without active components.

(3) Placing the mold containing the microneedle roots and the microneedle base from step (2) in a drying box for drying.

(4) After removing the mold from the drying box, peeling off an entire microneedle sheet from the mold.

(5) Attaching an adhesive tape to the back of the microneedle sheet, to obtain the microneedle patch.

(6) Packaging and sterilization.

### Embodiment 31: GH-Containing Soluble Microneedle Formulation and Microneedle Patch

The microneedle body includes a soluble microneedle formulation containing GH, including the following components in parts by weight: 3 parts of small molecule sodium hyaluronate, 3 parts of GH, and 94 parts of deionized water.

The microneedle roots and the microneedle base are composed of the soluble microneedle formulation with 0% content of active component, including the following components in parts by weight: 5 parts of large molecule polyvinylpyrrolidone, 25 parts of small molecule sodium hyaluronate, 5 parts of ginsenoside, and 65 parts of deionized water.

Where, the molecular weight of large molecule polyvinylpyrrolidone is ≥50,000 Da, and the molecular weight of small molecule sodium hyaluronate is <50,000 Da.

The preparation method of the GH-containing soluble microneedle formulation in this embodiment includes the following steps:

(1) Preparation of the microneedle body formulation: mixing 3 parts of small molecule sodium hyaluronate, 3 parts of GH, and 94 parts of deionized water, stirring and dissolving evenly to form solution A.

(2) Preparation of the microneedle formulation: mixing 5 parts of large molecule polyvinylpyrrolidone, 25 parts of small molecule sodium hyaluronate, 5 parts of ginsenoside, and 65 parts of deionized water, stirring and dissolving evenly to form solution B.

The preparation method of the GH-containing microneedle patch in this embodiment includes the following steps:

(1) Using high-pressure infusion to infuse solution A containing GH into a mold to form the microneedle tips with active components.

(2) Using a leveling method to infuse solution B into the mold containing the microneedle tips in step (1) to form the microneedle roots and the microneedle base without active components.

(3) Placing the mold containing the microneedle roots and the microneedle base in step (2) in a drying box to dry.

(4) After removing the mold from the drying box, peeling off an entire microneedle sheet from the mold.

(5) Attaching an adhesive tape on the back of the microneedle sheet to obtain the microneedle patch.

(6) Packaging and sterilization.

### Embodiment 32: Soluble Microneedle Formulation and Microneedle Patch Containing Inactivated Influenza Vaccines

The microneedle body includes a soluble microneedle formulation containing inactivated influenza vaccines, including the following components in parts by weight: 2 parts of inactivated influenza vaccines, 3 parts of small molecule sodium hyaluronate, and 95 parts of deionized water.

The microneedle roots and the microneedle base are composed of soluble microneedle formulation with 0% content of active component, including the following components in parts by weight: 5 parts of ginsenoside, 10 parts of large molecule polyvinylpyrrolidone, 20 parts of small molecule sodium hyaluronate, and 65 parts of deionized water.

Where, the molecular weight of large molecule polyvinylpyrrolidone is ≥50,000 Da, and the molecular weight of small molecule sodium hyaluronate is <50,000 Da.

In this embodiment, the preparation method of the soluble microneedle formulation containing inactivated influenza vaccines includes the following steps:

(1) Preparation of the microneedle body formulation: mixing 2 parts of inactivated influenza vaccines, 3 parts of small molecule sodium hyaluronate, and 95 parts of deionized water, stirring and dissolving evenly to form solution A.

(2) Preparation of the microneedle formulation: mixing 5 parts of ginsenoside, 10 parts of large molecule polyvinylpyrrolidone, 20 parts of small molecule sodium hyaluronate, and 65 parts of deionized water, stirring and dissolving evenly to form solution B.

In this embodiment, the preparation method of the microneedle patch containing inactivated influenza vaccines includes the following steps:

(1) Using a vacuum method to infuse solution A containing inactivated influenza vaccines into a mold to form the microneedle tips with active components.

(2) Using a leveling method to infuse solution B into the mold containing the microneedle tips from step (1) to form the microneedle roots and the microneedle base without active components.

(3) Placing the mold containing the microneedle roots and the microneedle base from step (2) in a drying box for drying.

(4) After removing the mold from the drying box, peeling off an entire microneedle sheet from the mold.

(5) Attaching an adhesive tape to the back of the microneedle sheet, to obtain the microneedle patch.

(6) Packaging and sterilization.

### Embodiment 33: Soluble Microneedle Formulation and Microneedle Patch Containing SARS-CoV-2 Vaccines

The soluble microneedle formulation containing SARS-CoV-2 vaccines includes the following components in parts by weight: 5 parts of large molecule sodium hydroxyethyl cellulose, 20 parts of small molecule sodium hyaluronate, 3 parts of small molecule trehalose, 0.5 part of SARS-CoV-2 vaccine, 2 parts of morpholinium salt, and 69.5 parts of deionized water.

Where, the molecular weight of large molecule sodium hydroxyethyl cellulose is ≥50,000 Da, and the molecular weights of small molecule sodium hyaluronate and small molecule trehalose are <50,000 Da.

In this embodiment, the preparation method of the soluble microneedle formulation containing SARS-CoV-2 vaccines includes the following steps:

(1) Mixing 30 parts of deionized water, 5 parts of large molecule sodium hydroxyethyl cellulose, 20 parts of small molecule sodium hyaluronate, and 3 parts of small molecule trehalose, stirring and dissolving evenly to form solution A.

(2) Adding a SARS-CoV-2 vaccine and a morpholinium salt to 39.5 parts of deionized water, stirring and dissolving evenly to form solution B.

(3) Mixing solution A and solution B, stirring, and dissolving evenly to obtain the soluble microneedle formulation containing SARS-CoV-2 vaccines.

In this embodiment, the preparation method of the microneedle patch containing SARS-CoV-2 vaccines includes the following steps:

(1) Using a high-pressure infusion method to infuse the microneedle formulation into a mold.

(2) Placing the mold in a drying box for drying.

(3) After removing the mold from the drying box, peeling off an entire microneedle sheet from the mold.

(4) Attaching an adhesive tape on the back of the microneedle sheet to obtain the microneedle patch.

(5) Packaging and sterilization.

### Embodiment 34: Soluble Microneedle Formulation and Microneedle Patch Containing DNA Vaccines

The soluble microneedle formulation containing DNA vaccines includes the following components in parts by weight: 10 parts of large molecule polyvinylpyrrolidone, 10 parts of small molecule sodium hyaluronate, 10 parts of small molecule collagen, 10 parts of small molecule sodium hydroxyethyl cellulose, 2 parts of DNA vaccines, 3 parts of pyridinium salts, and 55 parts of deionized water.

Where, the molecular weight of large molecule polyvinylpyrrolidone is ≥50,000 Da, and the molecular weights of small molecule sodium hyaluronate, small molecule sodium hydroxyethyl cellulose, and small molecule collagen are all <50,000 Da.

In this embodiment, the preparation method of the DNA vaccine-containing soluble microneedle formulation includes the following steps:

(1) Mixing 30 parts of deionized water, 10 parts of large molecule polyvinylpyrrolidone, 10 parts of small molecule sodium hyaluronate, 10 parts of small molecule collagen, and 10 parts of small molecule sodium hydroxyethyl cellulose, stirring, and dissolving evenly to form solution A.

(2) Adding DNA vaccines and pyridinium salts to 25 parts of deionized water, stirring, and dissolving evenly to form solution B.

(3) Mixing solution A and solution B, and stirring evenly to form a DNA vaccine-containing soluble microneedle formulation.

In this embodiment, the preparation method of the DNA vaccine-containing microneedle patch includes the following steps:

(1) Using a high-pressure infusion method to infuse the microneedle formulation into a mold;

(2) Placing the mold in a drying box for drying.

(3) After removing the mold from the drying box, peeling off an entire microneedle sheet from the mold.

(4) Attaching an adhesive tape on the back of the microneedle sheet to obtain the microneedle patch.

(5) Packaging and sterilization.

### Embodiment 35: Soluble Microneedle Formulation and Microneedle Patch Containing Hepatitis B Virus Vaccines

The soluble microneedle formulation containing hepatitis B virus vaccines includes the following components in parts by weight: 15 parts of large molecule polyvinylpyrrolidone, 10 parts of small molecule sodium hyaluronate, 20 parts of small molecule chitosan, 0.5 parts of hepatitis B virus vaccines, 5 parts of ginsenoside, and 49.5 parts of deionized water.

Where, the molecular weight of large molecule polyvinylpyrrolidone is ≥50,000 Da, and the molecular weights of small molecule sodium hyaluronate and small molecule chitosan are both <50,000 Da.

In this embodiment, the preparation method of the soluble microneedle formulation containing hepatitis B virus vaccines includes the following steps:

(1) Mixing 30 parts of deionized water, 15 parts of large molecule polyvinylpyrrolidone, 10 parts of small molecule sodium hyaluronate, and 20 parts of small molecule chitosan, stirring, and dissolving evenly to form solution A.

(2) Adding hepatitis B virus vaccines and ginsenoside to 19.5 parts of deionized water, stirring, and dissolving evenly to form solution B.

(3) Mixing solution A and solution B, and stirring evenly to obtain a soluble microneedle formulation containing hepatitis B virus vaccines.

In this embodiment, the preparation method of microneedle patch containing hepatitis B virus vaccines includes the following steps:

(1) Using a high-pressure infusion method to infuse the microneedle formulation into a mold;

(2) Placing the mold in a drying box for drying.

(3) After removing the mold from the drying box, peeling off an entire microneedle sheet from the mold.

(4) Attaching an adhesive tape on the back of the microneedle sheet to obtain the microneedle patch.

(5) Packaging and sterilization.

### Embodiment 36: Soluble Microneedle Formulation and Microneedle Patch Containing mRNA Vaccines

The soluble microneedle formulation containing mRNA vaccines includes the following components in parts by weight: 8 parts of large molecule sodium hyaluronate, 5 parts of small molecule sodium hyaluronate, 10 parts of dextran, 5 parts of sucrose, 5 parts of collagen, 2 parts of mRNA vaccines, 4 parts of morpholinium salts, and 61 parts of deionized water.

Where, the molecular weight of large molecule sodium hyaluronate is ≥50,000 Da, and the molecular weights of small molecule sodium hyaluronate, small molecule dextran, small molecule sucrose and small molecule collagen are all <50,000 Da.

In this embodiment, the preparation method of the soluble microneedle formulation containing mRNA vaccines includes the following steps:

(1) Mixing 30 parts of deionized water, 8 parts of large molecule sodium hyaluronate, 5 parts of small molecule sodium hyaluronate, 10 parts of small molecule dextran, 5 parts of small molecule sucrose, and 5 parts of small molecule collagen, stirring, and dissolving evenly to form solution A.

(2) Adding the mRNA vaccines and astragalus saponins to 31 parts of deionized water, stirring thoroughly, and dissolving evenly to form solution B.

(3) Mixing solution A and solution B, and stirring evenly to obtain the soluble microneedle formulation containing hepatitis B virus vaccines.

In this embodiment, the preparation method of the hepatitis B virus vaccine-containing microneedle patch includes the following steps:

(1) Using a high-pressure infusion method to infuse the microneedle formulation into a mold.

(2) Placing the mold in a drying box for drying.

(3) After removing the mold from the drying box, peeling off an entire microneedle sheet from the mold.

(4) Attaching an adhesive tape on the back of the microneedle sheet to obtain the microneedle patch.

(5) Packaging and sterilization.

The results of the microneedle patches prepared in Embodiments 21-36 in terms of needle deficiency rate, unflatness rate, and yield rate are shown in Table 3.

TABLE 3
Quality of microneedle patches

| | Needle deficiency | | |
|---|---|---|---|
| | rate % | Unflatness rate % | Yield rate % |
| Embodiment 21 | 13.9 | 2.1 | 84.0 |
| Embodiment 22 | 9.5 | 0 | 90.5 |
| Embodiment 23 | 8.6 | 1.3 | 90.1 |
| Embodiment 24 | 8.5 | 0.8 | 90.7 |
| Embodiment 25 | 4.2 | 1.6 | 94.2 |
| Embodiment 26 | 8.0 | 0 | 92.0 |
| Embodiment 27 | 7.3 | 0 | 92.7 |
| Embodiment 28 | 5.4 | 0 | 94.6 |
| Embodiment 29 | 6.2 | 0 | 93.8 |
| Embodiment 30 | 6.5 | 0 | 93.5 |
| Embodiment 31 | 6.0 | 0 | 94 |
| Embodiment 32 | 6.3 | 1.5 | 92.2 |
| Embodiment 33 | 2.1 | 0 | 97.9 |
| Embodiment 34 | 4.8 | 2 | 93.2 |
| Embodiment 35 | 5.2 | 3 | 91.8 |
| Embodiment 36 | 5.5 | 0 | 94.5 |

As shown in the above table, on the basis of the microneedle formulation composed of the aforementioned microneedle scaffold material and surfactants with a cyclic structure, an appropriate amount of active component is added. For active components that are insoluble or poorly soluble in water, mixing them with surfactants with a cyclic structure, and then mixing them with the microneedle scaffold solution, the surfactants with a cyclic structure in this preparation method can promote better solubility and stability of the active components that are insoluble or poorly soluble in water. Therefore, overall, it does not affect the presentation of the technical effects of the present disclosure. The prepared microneedle patch has a needle deficiency rate of 2.1-13.9%, an unflatness rate lower than 2.1%, and a yield rate of 84-97.9%. Compared with microneedle patches prepared without active components, there is no significant difference in the yield rate.

In order to further understand the technical effects of the present disclosure, the inventors provided some comparative experiments involving poorly soluble active components. The above comparison is only a part of the experiments and is not exhaustive.

### Comparative Example 9: Soluble Microneedle Formulation and Microneedle Patch for Removing Eye Bags

The difference between this comparative example and Embodiment 24 is that deionized water is used instead of aesculus saponins in the soluble microneedle formulation for removing eye bags.

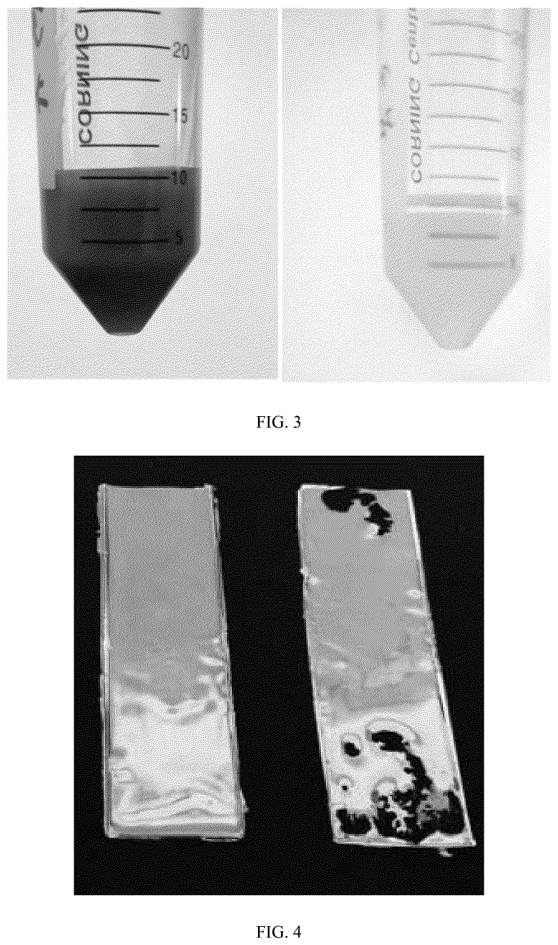

As shown in FIG. 2, when the skincare components include niacinamide and caffeine, where caffeine is a poorly soluble substance, in Embodiment 24, the addition of surfactants with cyclic structures prevents crystalline precipitation (seen in the right tube in FIG. 2). In contrast, in Comparative Example 9, a significant amount of caffeine crystals precipitated from the solution (seen in the left tube in FIG. 2).

This indicates that surfactants with cyclic structures have emulsifying abilities, enhancing the solubility of raw material and improving the dissolution and stability of other components that are insoluble or poorly soluble in water. As a result, the yield rate of the product is increased.

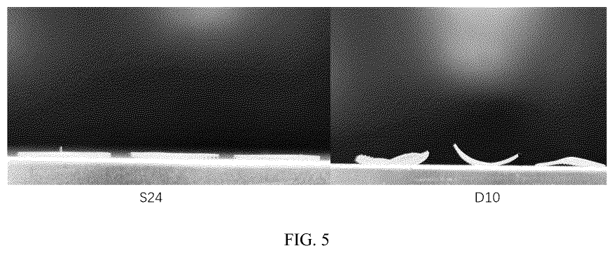

Additionally, as shown in FIG. 4, the microneedle patch prepared in Embodiment 24 exhibits almost no needle defects (seen on the left side of FIG. 4), while the microneedle patch prepared in Comparative Example 9 shows severe needle defects (seen on the right side of FIG. 4).

### Comparative Example 10: Soluble Microneedle Formulation and Microneedle Patch for Removing Eye Bags

The difference between Comparative Example 10 and Embodiment 24 is that fatty acyl diethanolamine is used instead of aesculus saponins in the soluble microneedle formulation for removing eye bags.

The needle deficiency rate of the microneedle patch prepared in Comparative Example 10 is 9.6%, which is not significantly different from that of Embodiment 24. However, due to the linear structure of fatty acyl diethanolamine surfactant compared to the cyclic structure of the surfactant in Embodiment 24, the moisture loss on the surface of the microneedle patch is uneven during the drying process, resulting in a significant decrease in the flatness of the microneedle patch. As shown in FIG. 5, the unflatness rate is as high as 56.3%.

### Comparative Example 11: Anti-Aging Soluble Microneedle Formulation and Microneedle Patch

The difference between this comparative example and Embodiment 25 is that deionized water is used instead of diosgenin in the anti-aging soluble microneedle formulation.

As can be seen from FIG. 3, the skin care components of Embodiment 25 are oily substances (tocopherol and retinol). When these active components are mixed with surfactants having cyclic structures, centrifugation and standing do not result in stratification, indicating better stability (as seen in the right tube of FIG. 3). While, in Comparative Example 11, where surfactants with cyclic structures are absent, stratification occurs after centrifugation and standing (as shown in the left tube of FIG. 3). This indicates that surfactants with cyclic structures act as surfactants, altering the surface tension of the target solution. They possess fixed hydrophilic and lipophilic groups that can be directionally arranged on the surface of the solution, thereby providing better stability for oily components in aqueous solutions.

In summary, the present disclosure utilizes surfactants with cyclic structures, which exhibit excellent compatibility with various microneedle scaffold materials, making them widely applicable. Additionally, these surfactants with cyclic structures have emulsifying ability, which can improve the solubility of raw materials, allowing other materials that are insoluble or poorly soluble in water to obtain higher solubility and better stability, thereby increasing the yield rate of the product.

Furthermore, in the segmented microneedle patch of the present disclosure, the microneedle formulation is utilized to prepare the microneedle roots and/or the microneedle base, which effectively reduces the needle deficiency rate and unflatness rate, thereby significantly improving the quality of the microneedle patch products. Moreover, the active components are concentrated on the microneedle bodies, especially the microneedle tips, so that the dosage of active components can be precisely controlled, leading to better efficacy.

It should be emphasized that the embodiments described in the present disclosure are illustrative rather than restrictive. Therefore, the present disclosure includes, but is not limited to, the embodiments described in specific implementations. Any other embodiments derived by those skilled in the art based on the technical solution of the present disclosure are also within the scope of protection of the present disclosure.

1. A microneedle formulation, comprising following compositions in mass percentage:
15-55% of microneedle scaffold material;
0.05-5% of surfactant with cyclic structure;
0-15% of active component; and
remaining being deionized water.

2. The microneedle formulation according to claim 1, wherein the surfactant with cyclic structure is at least one of a pyridinium salt, a morpholinium salt, an imidazolinium salt, a piperazinium salt, and a saponin; and
the microneedle scaffold material is at least one of sodium hyaluronate, polyvinylpyrrolidone, chondroitin sulfate, sodium hydroxyethyl cellulose, collagen, sucrose, trehalose, maltose, chitosan, and dextran.

3. The microneedle formulation according to claim 2, wherein the saponin is at least one of soapberry saponin, ginsenoside, astragalus saponin, aesculus saponin, diosgenin, and ruscogenin; and
the microneedle scaffold material comprises a mixture of large molecule microneedle scaffold material and small molecule microneedle scaffold material in a mass ratio of 1:1-3:1, or the microneedle scaffold material comprises a mixture of large molecule microneedle scaffold material and small molecule microneedle scaffold material in a mass ratio of 0:1-0.5:1.

4. The microneedle formulation according to claim 3, wherein a molecular weight of the large molecule microneedle scaffold material is ≥50,000 Da; and
a molecular weight of the small molecule microneedle scaffold material is <50,000 Da.

5. The microneedle formulation according to claim 1, wherein the active component is a drug, a vaccine, or a skincare component, wherein:
the drug comprises GLP-1 analogues, parathyroid hormone analogues, GH, donepezil, triamcinolone acetonide, or doxorubicin;
the vaccine comprises an inactivated influenza vaccine, a hepatitis B virus vaccine, a SARS-COV-2 vaccine, or an mRNA vaccine; and
the skincare component comprises at least one of a whitening component, a moisturizing component, an antioxidant component, an eye bag removing component, an anti-aging component, and a vascular color reducing component.

6. The microneedle formulation according to claim 5, wherein the whitening component comprises at least one of glabridin, niacinamide, phloretin, and phenethyl resorcinol;
the moisturizing component comprises at least one of glycerin and tetrahydromethylpyrimidine carboxylic acid;
the antioxidant component comprises at least one of glucosyl hesperidin and vitamin C ethyl ether;
the eye bag removing component comprises at least one of niacinamide and caffeine; and
the anti-aging component comprises at least one of tocopherol and retinol.

7. A method for preparing a microneedle formulation, wherein the microneedle formulation comprises 15-55% of microneedle scaffold material, 0.05-5% of surfactant with cyclic structure, 0-15% of active component, and remaining being deionized water, the method comprising:
dissolving the microneedle scaffold material, the surfactant with cyclic structure, and the active component in the deionized water, stirring evenly, and degassing, to obtain the microneedle formulation; or
dissolving the microneedle scaffold material in a portion of deionized water, and stirring evenly to obtain a scaffold solution; adding the surfactant with cyclic structure and the active component into a remaining portion of deionized water, and stirring evenly to obtain an active solution; and mixing the scaffold solution with the active solution, stirring evenly, and degassing, to obtain the microneedle formulation.

8. A microneedle patch, made from a microneedle formulation wherein the microneedle formulation comprises 15-55% of microneedle scaffold material, 0.05-5% of surfactant with cyclic structure, 0-15% of active component, and remaining being deionized water.

9. A method for preparing the microneedle patch according to claim 8, comprising:
infusing the microneedle formulation into a mold, and drying the mold;
removing the dried microneedle from the mold to obtain a microneedle sheet; and
attaching an adhesive tape to a back of the microneedle sheet, thereby obtaining the microneedle patch.

10. A method for preparing the microneedle patch according to claim 8, comprising:
infusing a first microneedle formulation containing the active component into a mold to form microneedle bodies or microneedle tips;
infusing a second microneedle formulation without the active component into the mold containing the microneedle bodies or the microneedle tips to form microneedle roots and/or a microneedle base;
drying and demolding the mold containing the microneedle roots and/or the microneedle base to obtain a microneedle sheet; and
attaching an adhesive tape to a back of the microneedle sheet, thereby obtaining the microneedle patch.
